(19) United States
(12) Patent Application Publication
(10) Pub. No.: US 2019/0164639 A1
(43) Pub. Date: May 30, 2019

(54) Automated Pharmaceutical Organizer and Dispenser

(71) Applicant: Vijay Saaraswat, Morganville, NJ (US)

(72) Inventor: Vijay Saaraswat, Morganville, NJ (US)

(21) Appl. No.: 15/654,582

(22) Filed: Jul. 19, 2017

(51) Int. Cl. G16H 20/13; G06F 16/23; G05B 15/02

(52) U.S. Cl. CPC G16H 20/13; G05B 15/02; G06F 16/2365

(57) ABSTRACT

In some embodiments, an automated pharmaceutical organizer and dispenser (“the dispenser”) is provided, which may comprise a computerized compartmentalized medicine sample or pharmaceutical dispenser designed for use by physician practices. In further embodiments, the dispenser may comprise a compartmentalized cabinet with control module configured to control vending of boxes of medicine samples from their respective compartments or impellers (eg. twisting helical springs) The control module that controls the cabinet is not necessarily attached to the cabinet and may control the dispensing of the sample boxes and also keeps an inventory of the same. The cabinet may communicate with the computer via Bluetooth, Wi-Fi, or have a physical cable connection. The control module may be attached to a printer where an inventory and other information can be printed.

### FIELD OF THE INVENTION

The present invention is in the technical field of computerized medicine sample organizer (“app”) and dispenser.

### BACKGROUND OF THE INVENTION

There exists a need for a Medicine Sample or Pharmaceutical Organizer and Dispenser, which is able to ease the task of dispensing pharmaceuticals by the physician. In a typical medicine storage unit, such as used in a hospital, the physician is required to reach in and grab the medicine from a bin, which is not much better than keeping pharmaceuticals on a closet shelf. It is important for the medicine to be dispensed individually, one at a time, and in a controlled fashion, however, currently available systems simply allow access to a bin full of medicine packages do not control for physicians taking more than one.

### SUMMARY OF THE INVENTION

In some embodiments, an automated pharmaceutical organizer and dispenser (“the dispenser”) is provided, which may comprise a computerized compartmentalized medicine sample or pharmaceutical dispenser designed for use by physician practices. In further embodiments, the dispenser may comprise a compartmentalized cabinet with control module configured to control vending of boxes of medicine samples from their respective compartments. In another embodiment the compartments may be comprised of a dispensing mechanism that keeps the pharmaceuticals separate but not physically compartmentalized per se. An example of this would be the use of twisting helical springs that rotate and propel the pharmaceuticals forward, at the same time keeping them separate, much as in ordinary vending machines. The control module that controls the cabinet is not necessarily attached to the cabinet and may optionally comprise a pc running control software that controls the vending of the sample boxes and also keeps an inventory of the same. The cabinet may communicate with the computer via Bluetooth, Wi-Fi, or have a physical cable connection. The control module may be attached to a printer where an inventory and other information can be printed.

In further embodiments, the dispenser is configured to dispense medicine samples or pharmaceuticals, which are usually boxed or packaged with multiple doses and the dispenser can be controlled by computer software, optionally not attached to the dispenser. The manner of dispensing may be similar to a vending machine, where a door opens and the pharmaceutical product may be dispensed. This will greatly ease the task of the physician, whereas in a typical medicine storage unit such as used in a hospital he or she will have to reach in and grab the medicine from a bin, which is not much better than keeping them on a closet shelf in the first place. In preferred embodiments, the dispenser may dispense the medicine packages individually, one at a time, and in a controlled fashion. Systems that simply allow access to a bin full of medicine packages do not control or prevent physicians from taking more than one package. This results in confounding any attempt to keep an accurate computerized inventory. Lastly, the cabinet may be lockable or locked by default, which will provide security and inventory control. The control module itself may optionally be attached to a printer.

The software of the control module will be important, and may be configured to keep inventory counts as well as brand name (or generic name), manufacturer, expiration dates, source of medicine (e.g. name of medicine company representatives). It can also warn when supply is running low, as well as display any other pertinent information. The software may give a graphical representation of inventory, such as bar or pie charts, and may give graphical representation of the pharmaceuticals with pictures of the tablets and/or the manufacture logo, for example. The control module preferably may be attached to a printer, where inventory lists and other information can be printed out. It will be a user-friendly software that may run on a desktop, or may be an application (“app”) to run on portable device operating systems.

The control software or “app” may stand on its own without a “dispenser” and may simply catalog the location of medicine on labelled shelves in a doctor's supply closet.

The application would have different users:

#1 Medical Representative. Medical representatives will have her their own login and password. The medical representative will be able to put medication samples into physician's closets or dispenser using the application. The medical representative will be able to document the number of medication samples, vouchers, coupons and any other relevant items into the application. These items will be available to review by physician immediately.

#2 Physician. The physician would be able to view his entire sample closet inventory into that application. This inventory is generated by the medical representative(s). The physician would be able to issue to the patient and if needed document patient's name and relevant information into that application. Once the inventory falls to a certain level, which is preset, the medical representative would get an alert via email or text message or by any other means. Multiple reports including sample distribution by medical representative, sample distribution by physician, etc. would be available. And further reports as deemed necessary would be able to be derived from application.

In further preferred embodiments, the novel dispenser disclosed may be configured to provide a very useful function for physicians who have many medicine samples or pharmaceuticals and wish to have better control over their supplies. Also, the dispenser may have a simple easy to use software interface that can operate from any pc and can control the cabinet remotely. An important feature of the software will be the ability to keep inventory of medicine sample boxes and expiration dates, source of medicine, drug warnings and any other pertinent information. The computerization of the pharmaceutical inventory will allow computer printing of inventory lists and other information. The dispenser may be lockable and maintain security of inventory housed within.

### BRIEF DESCRIPTION OF THE DRAWINGS

FIG. 1—FIG. 1 is a perspective view of an example of Automated Pharmaceutical Organizer and Dispenser according to various embodiments described herein.

FIG. 2—FIG. 2 is a side view of the exemplary Automated Pharmaceutical Organizer and Dispenser of FIG. 1 according to various embodiments described herein.

FIG. 3—FIG. 3 is a perspective view of an example of a vertical configuration of an Automated Pharmaceutical Organizer and Dispenser according to various embodiments described herein.

FIG. 4—FIG. 4 shows a flowchart of the control software that may installed on a desktop or mobile computing device (“app”).

FIG. 5—FIG. 5 shows flowchart of procedure to load pharmaceutical cabinet.

FIG. 6—FIG. 6 shows an example of a block diagram of a control module according to various embodiments described herein.

### DETAILED DESCRIPTION OF THE INVENTION

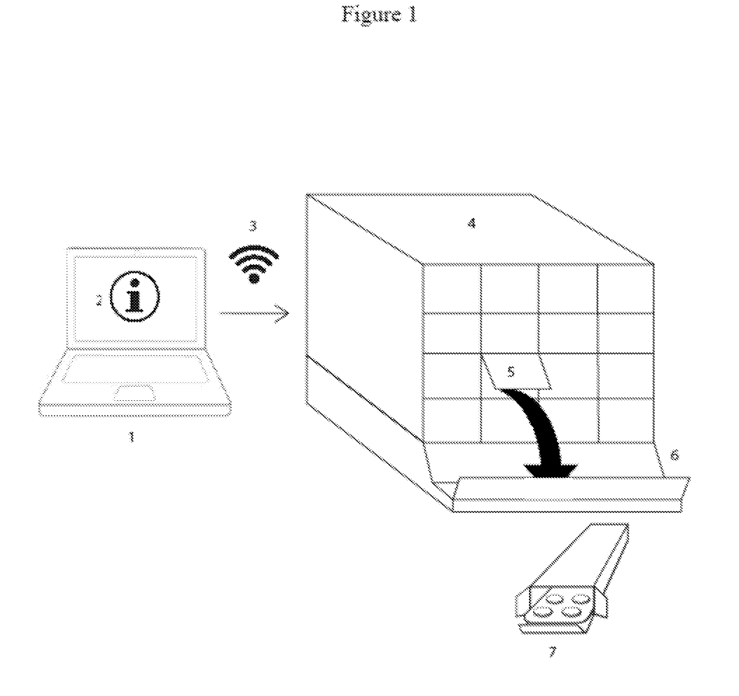

Referring now to the Automated Pharmaceutical Organizer and Dispenser (“the dispenser”) in more detail, in FIG. 1 there is shown to be a control module 1, which may comprise a personal computer, with control software 2 acting via a connection 3 (optionally Wi-Fi 3, wired, or blue tooth) upon a dispensing medicine cabinet 4. In some embodiments, the dispenser may comprise a cabinet 4, which may have one or more horizontal lengthwise compartments with a computer controlled door 5 configured to govern access to each compartment. Control module 1 may instruct the cabinet 4 to dispense a single box or package of medicine 7, which may optionally fall into a collecting bin 6, much like a vending machine. Software on control module 1 may keep track of inventory, manufacturer, lot and expiration date, etc. of the packages of medicine 7 within the cabinet 4. In other embodiments the compartments may be comprised of rows of helical springs driving the pharmaceutical forward, not physically “compartmentalized” per se, but organized and separate, again as in a vending machine.

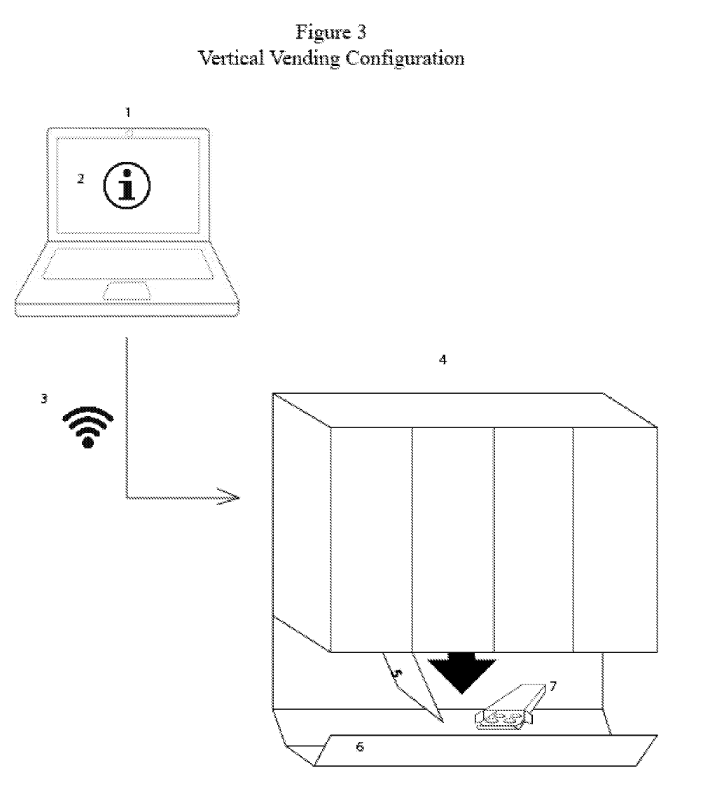

In further embodiments, the doors 5 of the storage compartments may vary in size to accommodate different sample boxes and package sizes, but could for example be 3 x 5 inches. Preferably, the doors 5 may be made of a clear plastic or other material to allow visualization of boxes being stored. The cabinet 4 and/or doors 5 may be made from any rigid material such as plastic, metal and metal alloys, wood, ceramic, resins, fiberglass, or any other suitable material. There will be a mechanism to allow for easy reloading of supplies, either through the back, or front of the cabinet. In some embodiments, if the mechanism is a helical spring that rotates and impels the product forward, the need for individual physical compartments may be obviated and the cabinet can function again much in the way of a vending machine. In all embodiments the packages are kept separate and organized. At the time of product loading, the newest pharmaceutical packages are loaded starting from the back so that they are dispensed in order of impending expiration. Also at the time of loading the number of product packages and their expiration date may be loaded into the database of the control software. In some embodiments, a cabinet 4 may be configured with a vertical configuration seen in FIG. 3 in which, for example, gravity may supply the dispensing force.

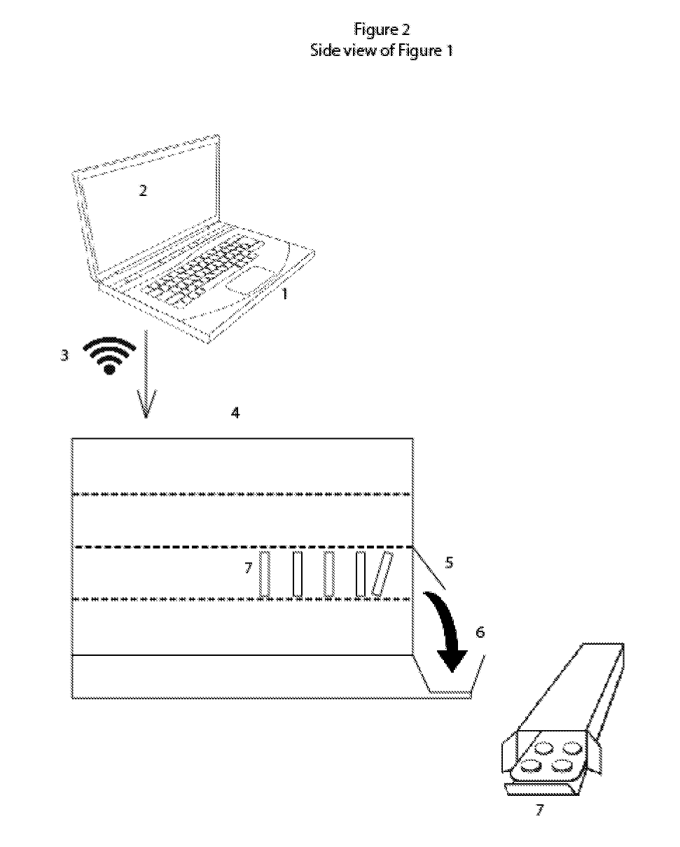

Referring now to the view shown in FIG. 2 the elements depicted in FIG. 1 are now shown in side view with the front of the dispensing cabinet 4 as drawn being to the right. In particular the boxes of medicine 7 are stored in a horizontal compartment, with control module 1 with software 2 commanding cabinet 4 via computer connection 3 to dispense said box of medicine 7 into the frontal mounted bin 6.

An alternative embodiment of the dispenser may include control module 1, software 2, and connection 3, but with a vertical orientation of the medicine storage compartments, as in FIG. 3. Analogous parts are numbered in FIGS. 1 and 2, namely control module 1, controlling/inventory software 2, wireless connection 3, dispensing cabinet 4, computerized opening of dispensing door 5, dispensing bin 6, and medication box being dispensed 7.

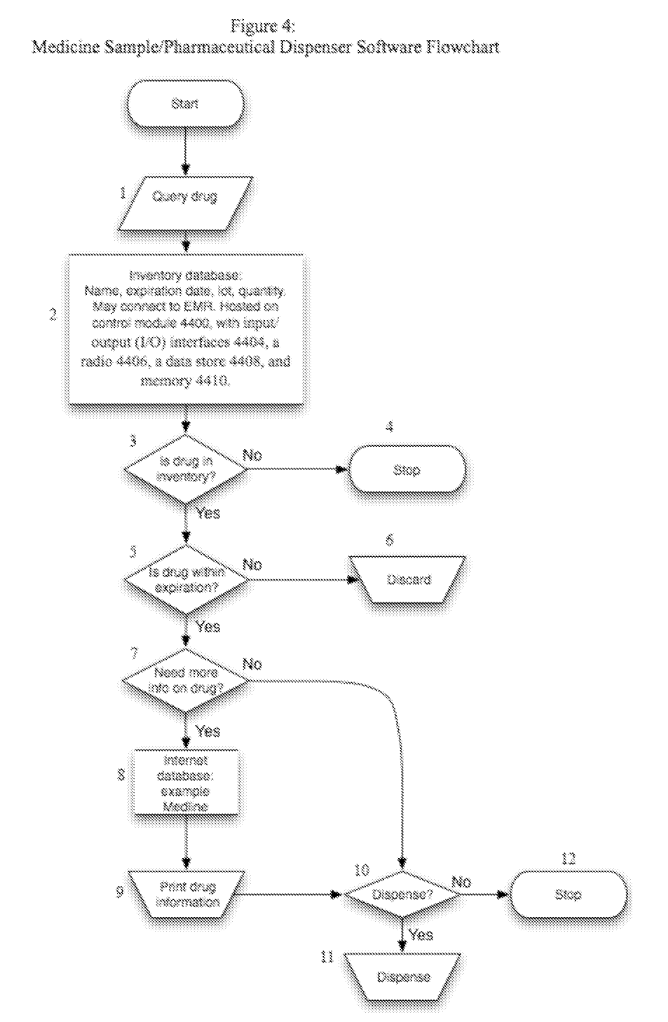

Referring to FIG. 4, in an exemplary embodiment, a flowchart demonstrates the dispensing function of the control software. This software will be installed on a control module, which may be an ordinary personal computer or a portable computing device such as a smartphone (as an “app”), which communicates with the pharmaceutical dispensing cabinet via Wi-Fi, Bluetooth or cable. The software accepts input, 1, of a drug name to be dispensed, which is then queried against a database, 2. The data in the database, 2, is separately entered at the time of pharmaceutical loading into the dispensing cabinet. The computer then checks if the pharmaceutical is in inventory, 3, is within expiration date, 5, and if the user wishes to know more about the pharmaceutical, 7. Depending on the results of these checks, the pharmaceutical is dispensed, 10, more information is printed, 9, or the user is informed the item is out of stock, 4, or expired, 6, and the program stops. The database, 2, may be augmented by connecting to the practice electronic medical records program (EMR). Or if incorporated into the EMR, the samples may be dispensed at the time of interaction with the patient at the operatory computer. To obtain additional information on a drug, 8, the software may communicate with an interne database. Before dispensing, 11, the user may enter the number of units to dispense. To obtain a different pharmaceutical the process is repeated from the beginning.

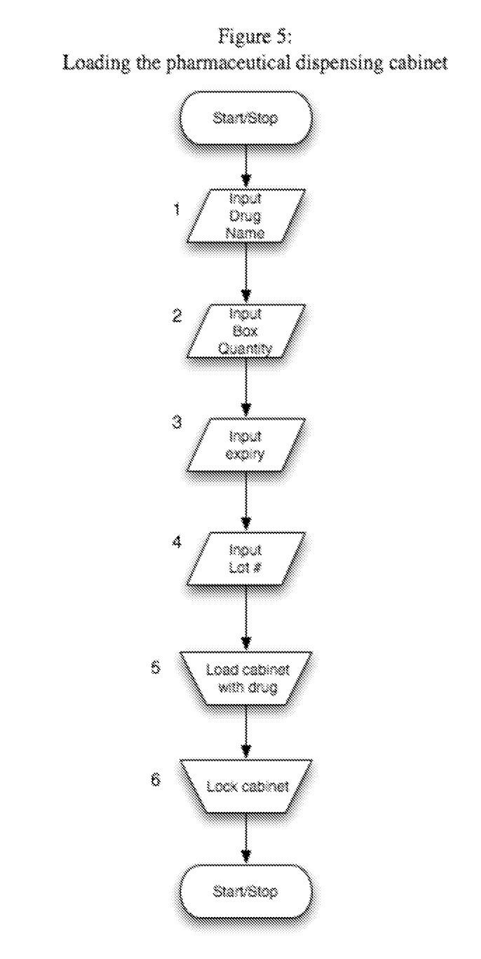

FIG. 5 illustrates the procedure for loading the pharmaceutical cabinet. The drug name, quantity, expiration date and lot number are entered into the control module in steps 1 through 4. The software may aid in the input of drugs by suggesting the most common or similar drugs while the data is being inputted (instant suggestion). Importantly in step 5 the drug packages are loaded into the cabinet starting from the back, such that the most imminently expiring medication is contained towards the front of the cabinet to be dispensed first.

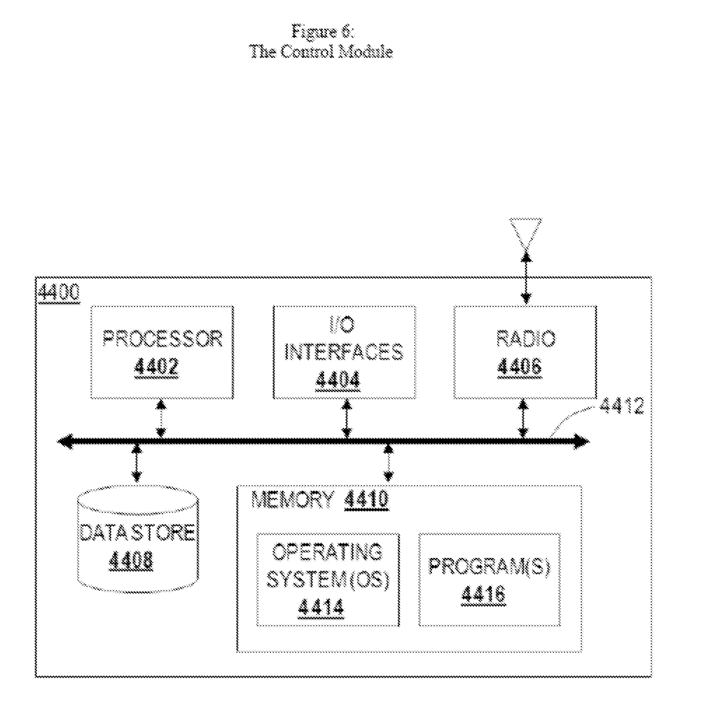

Referring to FIG. 6, in an exemplary embodiment, a block diagram illustrates a control module 4400, which may be used by the dispenser. The term “control module ” as used herein is a type of electronic device comprising circuitry and configured to generally perform functions such as recording audio, photos, and videos; displaying or reproducing audio, photos, and videos; storing, retrieving, or manipulation of electronic data; providing electrical communications and network connectivity; or any other similar function. Non-limiting examples of electronic devices include; personal computers (PCs), workstations, laptops, tablet PCs including the iPad, cell phones including iOS phones made by Apple Inc., Android OS phones, Microsoft OS phones, Blackberry phones, digital music players, or any electronic device capable of running computer software and displaying information to a user, memory cards, other memory storage devices, digital cameras, external battery packs, external charging devices, and the like. Certain types of electronic devices, which are portable and easily carried by a person from one location to another, may sometimes be referred to as a “portable electronic device” or “portable device”. Some non-limiting examples of portable devices include; cell phones, smart phones, tablet computers, laptop computers, wearable computers such as watches, Google Glasses, etc. and the like.

The control module 4400 can be a digital device that, in terms of hardware architecture, generally includes a processor 4402, input/output (I/O) interfaces 4404, a radio 4406, a data store 4408, and memory 4410. It should be appreciated by those of ordinary skill in the art that FIG. 6 depicts the control module 4400 in an oversimplified manner, and a practical embodiment may include additional components and suitably configured processing logic to support known or conventional operating features that are not described in detail herein. The components (4402, 4404, 4406, 4408, and 4410) are communicatively coupled via a local interface 4412. The local interface 4412 can be, for example but not limited to, one or more buses or other wired or wireless connections, as is known in the art. The local interface 4412 can have additional elements, which are omitted for simplicity, such as controllers, buffers (caches), drivers, repeaters, and receivers, among many others, to enable communications. Further, the local interface 4412 may include address, control, and/or data connections to enable appropriate communications among the aforementioned components.

The processor 4402 is a hardware device for executing software instructions. The processor 4402 can be any custom made or commercially available processor, a central processing unit (CPU), an auxiliary processor among several processors associated with the control module 4400, a semiconductor-based microprocessor (in the form of a microchip or chip set), or generally any device for executing software instructions. When the control module 4400 is in operation, the processor 4402 is configured to execute software stored within the memory 4410, to communicate data to and from the memory 4410, and to generally control operations of the electronic device 4400 pursuant to the software instructions. In an exemplary embodiment, the processor 4402 may include a mobile optimized processor such as optimized for power consumption and mobile applications. The I/O interfaces 4404 can be used to receive user input from and/or for providing system output. User input can be provided via, for example, a keypad, a touch screen, a scroll ball, a scroll bar, buttons, bar code scanner, and the like. System output can be provided via a display device such as a liquid crystal display (LCD), touch screen, and the like. The I/O interfaces 4404 can also include, for example, a serial port, a parallel port, a small computer system interface (SCSI), an infrared (IR) interface, a radio frequency (RF) interface, a universal serial bus (USB) interface, and the like. The I/O interfaces 4404 can include a graphical user interface (GUI) that enables a user to interact with the control module 4400. Additionally, the I/O interfaces 4404 may further include an imaging device, i.e. camera, video camera, etc.

The radio 4406 enables wireless communication to an external access device or network. Any number of suitable wireless data communication protocols, techniques, or methodologies can be supported by the radio 4406, including, without limitation: RF; IrDA (infrared); Bluetooth; ZigBee (and other variants of the IEEE 802.15 protocol); IEEE 802.11 (any variation); IEEE 802.16 (WiMAX or any other variation); Direct Sequence Spread Spectrum; Frequency Hopping Spread Spectrum; Long Term Evolution (LTE); cellular/wireless/cordless telecommunication protocols (e.g. 3G/4G, etc.); wireless home network communication protocols; paging network protocols; magnetic induction; satellite data communication protocols; wireless hospital or health care facility network protocols such as those operating in the WMTS bands; GPRS; proprietary wireless data communication protocols such as variants of Wireless USB; and any other protocols for wireless communication. The data store 4408 may be used to store data. The data store 4408 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 4408 may incorporate electronic, magnetic, optical, and/or other types of storage media.

The memory 4410 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 4410 may incorporate electronic, magnetic, optical, and/or other types of storage media. Note that the memory 4410 may have a distributed architecture, where various components are situated remotely from one another, but can be accessed by the processor 4402. The software in memory 4410 can include one or more software programs, each of which includes an ordered listing of executable instructions for implementing logical functions. In the example of FIG. 6, the software in the memory 4410 includes a suitable operating system (O/S) 4414 and programs 4416. The operating system 4414 essentially controls the execution of other computer programs, and provides scheduling, input-output control, file and data management, memory management, and communication control and related services. The programs 4416 may include various applications, add-ons, etc. configured to provide end user functionality with the electronic device 4400. For example, exemplary programs 4416 may include, but not limited to, a web browser, social networking applications, streaming media applications, games, mapping and location applications, electronic mail applications, financial applications, and the like. In a typical example, the end user typically uses one or more of the programs 4416 to control the dispensing of packages of pharmaceuticals by the dispenser.

The advantages of the present invention include, without limitation, ease of inventory of medicine samples for harried physicians in private practice, computerized dispensing and security of medicine sample storage at all times. Samples preferably are to be dispensed in a vending machine manner.

In some embodiments, the disclosed dispenser provides a computerized, medicine sample or pharmaceutical box dispenser that enables vending machine-like dispensing and ease as well as computerized inventory control and keeping of data such as lot number, expiry date, etc. Inventory and other data can be printed out by the control module 1.

In other embodiments the control software (“app”) may control the inventory, distribution and entering of pharmaceutical samples via a computerized database, that may reference locations on numbered shelves in a doctor's closet, thereby foregoing the “dispenser” altogether.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

1. A novel Computerized Pharmaceutical Organizer and Dispenser is claimed in this patent application and consists of a control software (or “app”) that catalogs and controls the dispensing of pharmaceuticals and pharmaceutical vouchers from a dispenser, or more simply, labelled shelves, and allows database entry of expiration dates, inventory, and medical representative names, with the user able to access and control this information at a glance.
